(19) United States
(12) Patent Application Publication
(10) Pub. No.: US 2025/0311915 A1
(43) Pub. Date: Oct. 9, 2025

(54) COMPACT ENDOSCOPE DESIGN FOR THREE-DIMENSIONAL SURGICAL GUIDANCE

(71) Applicant: THE JOHNS HOPKINS UNIVERSITY, Baltimore, MD (US)

(72) Inventors: Jin Ung Kang, Glenelg, MD (US); Hanh N.D. Le, Baltimore, MD (US)

(21) Appl. No.: 19/015,310

(22) Filed: Jan. 9, 2025

Related U.S. Application Data
(63) Continuation of application No. 15/674,553, filed on Aug. 11, 2017, now abandoned.
(60) Provisional application No. 62/374,267, filed on Aug. 12, 2016.

(51) Int. Cl. A61B 1/00; A61B 1/04; A61B 1/06; A61B 1/07; A61B 5/00; A61B 5/107

(52) U.S. Cl. CPC A61B 1/00194; A61B 1/00057; A61B 1/00064; A61B 1/00193; A61B 1/0605; A61B 1/0623; A61B 1/0627; A61B 1/07; A61B 5/1079; A61B 5/6852; A61B 1/044

(57) ABSTRACT

The present invention is directed to endoscopic structure illumination to provide simple, inexpensive 3D endoscopic technique to conduct high resolution 3D imagery for use in surgical guidance system. The present invention is directed to an FPP endoscopic imaging setup which provides a wide field of view (FOV) that addresses a quantitative depth information and can be integrated with commercially available endoscopes to provide tissue profilometry. Furthermore, by adapting a flexible camera calibration method for the 3D reconstruction technique in free space, the present invention provides an optimal fringe pattern for the inner tissue profile capturing within the endoscopic view and validate the method using both static and dynamic samples that exhibits a depth of field (DOF) of approximately 20 mm and a relative accuracy of 0.1% using a customized printed calibration board. The presented designs enable flexibility in controlling the deviated angle necessary for single scope integration using FPP method.

### CROSS REFERENCE TO RELATED APPLICATIONS

This application is a continuation of U.S. patent application Ser. No. 15/674,553, filed on Aug. 11, 2017, which claims the benefit of U.S. Provisional Patent Application No. 62/374,267, filed on Aug. 12, 2016, which is incorporated by reference herein, in its entirety.

### GOVERNMENT RIGHTS

This invention was made with government support under CBET-1430040 awarded by the National Science Foundation. The government has certain rights in the invention.

### FIELD OF THE INVENTION

The present invention relates generally to medical devices. More particularly, the present invention relates to a compact endoscope design for three-dimensional surgical guidance.

### BACKGROUND OF THE INVENTION

Surgeons have been increasingly utilizing minimally invasive surgical guidance techniques not only to reduce surgical trauma but also to achieve accurate and objective surgical risk evaluations. A typical minimally invasive surgical guidance system provides visual assistance in two-dimensional (2D) anatomy and pathology of internal organ within a limited field of view (FOV). Current developments in minimally invasive 3D surgical endoscope involve techniques such as stereoscopy, time of flight (ToF), and structure illumination to achieve depth discrimination.

3D stereoscopy is a well-developed technique which relies on the searching of stereo correspondence between two distinct views of the scene and produce disparity estimation from the both images. From the calculated disparities, the 3D structure can be deduced using triangulation method based on the geometric calibration with the camera. Different stereo reconstruction algorithms have been used to establish disparities both spatially and temporally for the correspondence extraction of the images from the both views to achieve depth resolution from 0.05 mm to 0.6 mm (the hybrid recursive matching), with flexibility in disparity extraction based on feature-based technique (the seed propagation method) or using an efficient convex optimization for disparity searching based on a 3D cost-volume configuration (the cost-volume method). These methods of 3D reconstruction has been commercially developed and have found wide acceptance throughout United States and Europe.

Time-of-flight method calculates travelling distance of light emitted from the illumination source and reflected by the object to the sensor, and deduces the depth information from the time difference between the emitted and reflected light, to reconstruct surface structure in 3D. Therefore, this technique does not rely on correspondence search or baseline restriction, leading to a compact design in use for surgical endoscopic system. Depth resolution using 3D time-of-fight surgical endoscope ranges from 0.89 mm to about 4 mm. However, due to the low light environment in tissue imaging, a ToF camera often uses high power laser source to illuminate internal targets. Some other limitations in depth evaluation using ToF come from specular reflectance, inhomogeneous illumination, and other systemic errors such as the sensor run-time, its temperature tolerance and imaging exposure time. Other tissue-light interaction factors such as tissue biological properties in absorption and scattering also contribute in the ToF systematic error. So far, an industrial prototype of the ToF for surgical endoscope (Richard Wolf GmbH (Knittlingen, Germany)) was introduced but the device has not yet been widely accepted.

Plenoptic imaging, or light field imaging, calculates depth from a single image collected by multiple reflected rays from the object through a microlens array located in front of a camera sensor. Each image from the sensor comprised of multiple micro images from the microlens array, i.e. each pixel of a micro image related to particular directions of different incoming light. The microlenses are aligned in a specific configuration and can come with different focusing lengths (multi-focus plenoptic camera) for an optimization of a maximal effective lateral resolution and the required depth of field. The lateral resolution of a multi-focus plenoptic camera can achieve up to a quarter of the sensor pixel in lateral resolution with an order of magnitude of 1 mm in axial resolution. Although benefiting from high depth resolution, 3D reconstruction using plenoptic imaging often requires customized sensor and microlens array for a particular imaging fields. Plenoptic imaging have also been commercially developed, primarily for consumer and non-medical applications.

Structured illumination or fringe projection profilometry (FPP) provides depth quantification similar to stereoscope technique, i.e. FPP relies on the parallax and triangulation of tissue location in relation to the camera and the projector. However, instead of searching for disparities, FPP detects the fringe patterns which are actively projected onto the tissue, therefore it highlights feature points presented in discontinuing surface or homogeneous regions. Moreover, the hardware flexibility also makes FPP simpler to implement compared to the abovementioned techniques. For in-vitro medical application, FPP has been used widely in dermatology for skin and wound inspection and health-care for idiopathic scoliosis diagnostic. However, FPP endoscopic imaging for medical intraoperative inspection has been so far rather limited. Certain efforts using FPP in laparoscope have been made. An example is FPP endoscope for augmented reality visualization, the device provides a direct depth perception overlaid capture on recorded images of trial phantom and tissue cadaver. Another example is in tubular tissue scanning, where the use of a collection of color light rings that achieved an average dimensional error of 92 μm. Majority of laparoscope development in tissue profilometry involves the use of a high power light source for SNR enhancement. Some examples such as in motion tracking using 100 mW, 660 nm wavelength laser diode and produces tracking error of 0.05 mm and for tissue profilometry with an accuracy of 0.15 mm using multispectral spot projection dispersed via a prism from a 4 W supercontinuum light source. Although these techniques achieve high precision and small cavity access, they often require complicated and may be prohibitively expensive hardware setup.

Accordingly, there is a need in the art for a compact, cost-effective endoscope design for three-dimensional surgical guidance.

### SUMMARY OF THE INVENTION

The foregoing needs are met, to a great extent, by the present invention which provides a device for 3D imagery including an imaging probe configured for fringe projection profilometry (FPP) and configured to provide a wide field of view (FOV). The device includes a CCD sensor. The device also includes an illumination probe and a digital projector.

In accordance with an aspect of the present invention, the device includes an angle controller. The angle controller sets a distance for separation between the imaging probe and the illumination probe. The device includes a housing for the imaging probe, the illumination probe, and the angle controller. The device also includes a non-transitory computer readable medium programmed to execute a flexible camera calibration method for the 3D reconstruction in free space to provide an optimal fringe pattern for an inner tissue profile.

In accordance with another aspect of the present invention, the imaging probe and the illumination probe each have a diameter of 5 mm. The device can also include a digital micromirror device (DMD). The DMD is configured to project a fringe pattern. There is a minimum of a 150 angle between the imaging and illumination probe. The device also includes synchronizing structured patterns from the DMD with the imaging camera.

In accordance with yet another aspect of the present invention, a method for a 3D image of a region of interest includes providing a wide field of view (FOV) with an imaging probe, such that quantitative depth information is addressed. The method includes illuminating the region of interest. The method also includes projecting a fringe pattern and capturing the 3D image of the region of interest.

In accordance with still another aspect of the present invention, the method includes using an illumination probe for illuminating the region of interest. The method includes setting a distance of separation between the imaging probe and the illumination probe. The method includes setting the distance of separation to be a minimum of a 15° angle. The method includes executing the method in conjunction with a non-transitory computer readable medium. The non-transitory computer readable medium is programmed to execute a flexible camera calibration method for 3D reconstruction in free space to provide an optimal fringe pattern for an inner tissue profile. The method includes using a digital micromirror device (DMD) to project the fringe pattern. The method includes using a coordinate transform to correspond each image point to each respective point on the DMD via the collected fringe patterns. The method includes using a CCD camera to capture the 3D image of the region of interest. The method can also include providing tissue profilometry.

### BRIEF DESCRIPTION OF THE DRAWING

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates an image view of an experimental setup, and FIG. 1B illustrates a schematic diagram of a fringe projection profilometry (FPP) endoscopic system, both according to an embodiment of the present invention.

FIG. 2A illustrates an image and graphical view of a depth of field target with its intensity profile across the dashed line, and FIG. 2B illustrates a graphical view of the corresponding height map.

FIGS. 3A and 3B illustrate image views of human dried temporal bone reveals cochlear section with its height map in 2D, as illustrated in FIG. 3A, and its corresponding 3D images at different section planes, as illustrated in FIG. 3B.

FIG. 4A illustrates image views of a human mouth cavity reflectance image with its corresponding height map when the inner cavity is close (above row) and open (below row), and FIG. 4B illustrates a height of segmented sections for both cases along the dashed lines.

FIG. 5 illustrates a schematic diagram of an embodiment with a flexible fiber-based endoscope. Region A is detailed in FIG. 7.

FIG. 6 illustrates a cross-sectional view of the tubing containing the fibers and angle-controller flexible probe.

FIG. 7 illustrates a partially sectional view of Region A of FIG. 7 with two angle control cases (when the wedge is closed and opened).

FIG. 8 illustrates a partially sectional view of another embodiment of region A of FIG. 7.

FIG. 9 illustrates a schematic diagram of another embodiment with two commercially available rigid scopes with a fixation adapter. Region B is detailed in FIG. 12.

FIG. 10 illustrates a partially sectional view of region B of FIG. 11).

FIG. 11 illustrates a schematic diagram of another embodiment with one rigid scope and a flexible medical graded scope with the angle controller.

FIG. 12 illustrates a partially sectional view of region C in FIG. 13.

FIG. 13 illustrates a partially sectional view of an extended wedge arm with multiple joints to access small space region.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to endoscopic structure illumination to provide a simple, inexpensive 3D endoscopic technique to conduct high resolution 3D imagery for use in surgical guidance system. The present invention is directed to an FPP endoscopic imaging setup which provides a wide field of view (FOV) that addresses a quantitative depth information and can be integrated with commercially available endoscopes to provide tissue profilometry. Furthermore, by adapting a flexible camera calibration method for the 3D reconstruction technique in free space, the present invention provides an optimal fringe pattern for the inner tissue profile capturing within the endoscopic view and validate the method using both static and dynamic samples that exhibits a depth of field (DOF) of approximately 20 mm and a relative accuracy of 0.1% using a customized printed calibration board.

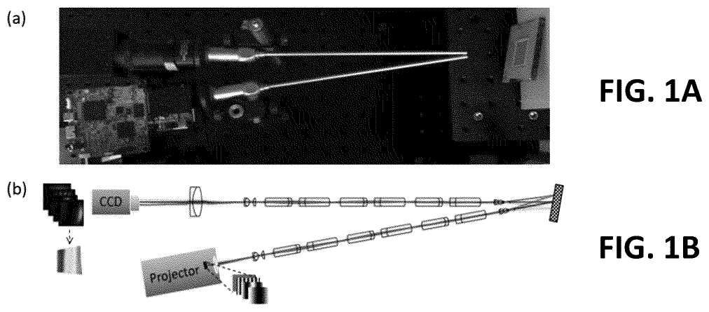

The fringe-projection-based 3D endoscopic system of the present invention, as illustrated in FIGS. 1A and 1B, consists of two identical traditional rigid surgical endoscopes (0-degree, 5-mm-diameter endoscope, Karl Storz GmbH & Co. KG, Tuttlingen, Deutschland) for illumination and imaging. The illumination endoscope guides the fringe patterns projected by a digital micromirror device (DMD) (TI-DLP EVM3000, Texas Instrument, Dallas, Texas, USA). The illumination light from the DMD is coupled into an illumination endoscope, and the fringe-pattern illuminated object is imaged via the secondary endoscope and a CCD camera (GS3-U3-15S5M-C, Point Grey Research Inc, Richmond, Canada) with an achromatic doublet lens (AC254-060-A, Thorlabs, Newton, New Jersey, USA). For a low-noise triangulation and to minimize the overall form-factor of the endoscope, a minimum angle of 15 degree between the illumination and the imaging scopes is chosen to maintain the desired field of view and height accuracy. The structured patterns were synchronized with the imaging camera to obtain high data rates and image quality. For the ray-tracing demonstration purpose, a standard endoscope model was adapted with the relay lens system based on a combination of rod lens. The system is controlled via a customized C#program on a Dell Precision T7600 workstation and the optical system is modelled using Zemax OpticsStudio 15 SP1 (Zemax, Kirkland, Washington, USA). FIG. 1A illustrates an image view of an experimental setup, and FIG. 1B illustrates a schematic diagram of a fringe projection profilometry (FPP) endoscopic system, both according to an embodiment of the present invention.

The FPP reconstruction method is based on parallax and triangulation between the camera and the structured light from the projector, in relation with the sample surface. In other words, a coordinate transform is used to correspond each image point on the camera sensor to each respective point on the DMD via the collected fringe patterns, i.e. treating the DMD as a second camera similar to the stereo-vision technique. To establish the transformation, a governing equation such as in Eq. (5) was derived to relate the object depth information to the phase map of the projection fringes. A vertical, frequency-shifted sinusoidal fringe wave as formulated in Eq. (1) is typically used to perform FPP to achieve full field and fast image processing. This fringe wave pattern is given as:

$${I}_{i} ( u , v ) = {I}_{o} [ 1 + cos ( \frac{2 π ku}{w} + δ ) ] . \qquad ( 1 )$$

where Iₒ is the intensity modulation amplitude, (u, v) are the spatial pixel indices, δ the shifted phase and k the fringe number, w the pattern width. In the application, k={1, 2, 6, 30}.

After projection of the pattern and collection of the images, the wrapped phase of each obtained image with different fringe patterns are calculated using the conventional four-step phase shift method as given by:

$$tan [ {ϕ}_{n}^{w} ( u , v ) ] = \frac{{I}_{4} ( u , v ) - {I}_{2} ( u , v )}{{I}_{1} ( u , v ) - {I}_{3} ( u , v )} . \qquad ( 2 )$$

where I₁₋₄ represents the intensity of shifted image.

In as much as the periodity of the collected structured patterns, its phase map at each point is restrained to a principal range, leading to the phase discontinuity at higher frequency fringes, therefore phase unwrapping is necessary to extract the absolute phase value. The phase unwrapping technique of the present invention is formulated based on the relation between the current and previous unwrapped phase information from the previous frequency, as described in Eq. (4), with the lowest frequency defined to have one fringe on its pattern, therefore its unwrapped phase is equal to its wrapped phase. For other higher frequencies, the unwrapped phase distribution can be calculated based on the unwrapped phase distribution of the previous frequency

$${ϕ}_{1} ( u , v ) = {ϕ}_{1}^{w} ( u , v ) . \qquad ( 3 )$$

$${ϕ}_{n} ( u , v ) = {ϕ}_{n}^{w} ( u , v ) + 2 π \cdot 〈 \frac{{ϕ}_{n - 1}^{u w} \frac{{f}_{n}}{{f}_{n - 1}} - {ϕ}_{n}^{w}}{2 π} 〉 . \qquad ( 4 )$$

where the wrapping operator [symbol] • [symbol] denotes the argument rounding to the closest integer, the superscript uw and w refers to unwrapped and wrapped, f represents fringe frequency, i.e. number of fringe per projection pattern and n={2, 3, 4} is the nth order of fringe frequency where

The out-of-plane height z at each pixel index (i,j) is proportional to the unwrapped phase as in Eq. (5):

$$z = \frac{\begin{matrix}{c}_{o} + {c}_{1} ϕ + ( {c}_{2} + {c}_{3} ϕ ) u + ( {c}_{4} + {c}_{5} ϕ ) v + ( {c}_{6} + {c}_{7} ϕ ) {u}^{2} + \\ ( {c}_{8} + {c}_{9} ϕ ) {v}^{2} +\end{matrix}}{\begin{matrix}{d}_{o} + {d}_{1} ϕ + ( {d}_{2} + {d}_{3} ϕ ) u + ( {d}_{4} + {d}_{5} ϕ ) v + ( {d}_{6} + {d}_{7} ϕ ) {u}^{2} + \\ ( {d}_{8} + {d}_{9} ϕ ) {v}^{2} + ( {d}_{10} + {d}_{11} ϕ ) uv\end{matrix}} . \qquad ( 5 )$$

where cₒ₋₁₁, dₒ₋₁₁ are constants determined by geometrical and other relevant parameters, ϕ is unwrapped phase distribution. The extension to second order of u and v is for the accuracy enhancement for complex real-world structures.

The calibration technique to determine cₒ₋₁₁, dₒ₋₁₁ was performed using a customized printed ring-centered calibration board. Its positions and tilting angles are varied to cover the interested imaging volume. Each calibration control point j at a board position i on the calibration board is transformed to the corresponding point (X_{c,ij}, Y_{c,ij}, Z_{c,ij}) in the camera coordinate system. The first board position (i=1) is determined to be the reference plane. A reference plane is the zero height of the 3D image structure and constructed by placing the calibration board perpendicular to the optical axis from the camera to the object. The reference plane is then formulated by fitting a planar equation with constant coefficient A, B, C to every point of the first board image.

$${z}_{ij} = \frac{{AX}_{c , ij} + {BY}_{c , ij} + {CZ}_{c , ij} + 1}{{A}^{2} + {B}^{2} + {C}^{2}} . \qquad ( 6 )$$

After the calculation of unwrapped phase ϕ and the height zᵢⱼ of each calibration control point j, the Levenberg-Marquard least-squares fitting method is used to obtain the coefficients cₒ₋₁₁, dₒ₋₁₁ in Eq. (5).

$${\sum }_{i = 1}^{a} {\sum }_{j = 1}^{b} {( z - {z}_{ij} )}^{2} . \qquad ( 7 )$$

To validate the 3D endoscope, several static and dynamic objects with structural complexity were imaged, such as a depth of field target (DOF 5-15. Edmund Optics, York, UK), a dried human temporal bone, and a human mouth cavity.

To measure the depth of field of the system, the intensity along a horizontal line pair section, was examined to determine the region of greatest contrast between line pairs. The DOF target was uniformly illuminated by a white LED light source (MCWHL1, Thorlabs, Newton, New Jersey, USA), and the target is located such that the first point of the target is in the same plane as the endoscopes' distal end. The optimal DOF is about 20 mm, i.e. images within 20 mm would give the best height accuracy.

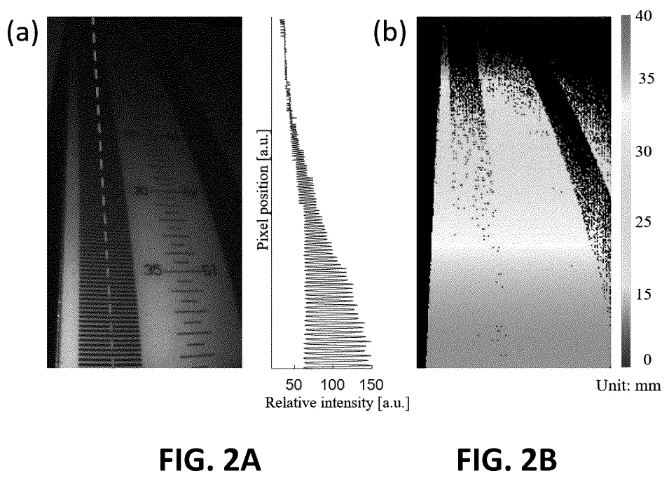

To minimize the specular reflectance on the DOF target, the DOF was moved 10 mm away from the distal end of the endoscopes. To calculate the height accuracy within the DOF range, which is indicated in FIGS. 2A and 2B and Table 1, the distance between horizontal lines on the DOF target is measured and compared. The mean calculated depth is the average of four measured depths within the compared depth range and is marked by the ruler on the DOF target. The error is the difference between the physical depth indicated by the ruler on the DOF target and the mean calculated depth, and the standard deviation measures the dispersion of the collected depth dataset. As shown in Table 1, the largest mean error is 43 μm and the largest standard deviation is 114 μm. With the entire FOV diameter measured to be 30.82 mm and the largest error lying on the mean calculated depth of 9.957 mm, the relative error is calculated by the ratio of the error over the total FOV as approximately 0.1%. FIG. 2A illustrates an image and graphical view of a depth of field target with its intensity profile across the dashed line, and FIG. 2B illustrates a graphical view of the corresponding height map.

TABLE 1
Comparison of physical and calculated
depth of the depth of field target

| | Mean calculated | | Standard |
|---|---|---|---|
| Physical depth | depth | Error | deviation |
| 0.5 | 0.499 | 0.001 | 0.008 |
| 1 | 0.994 | 0.006 | 0.042 |
| 2 | 2.017 | −0.017 | 0.035 |
| 5 | 5.039 | −0.039 | 0.114 |
| 10 | 9.957 | 0.043 | 0.087 |

Unit: millimeter

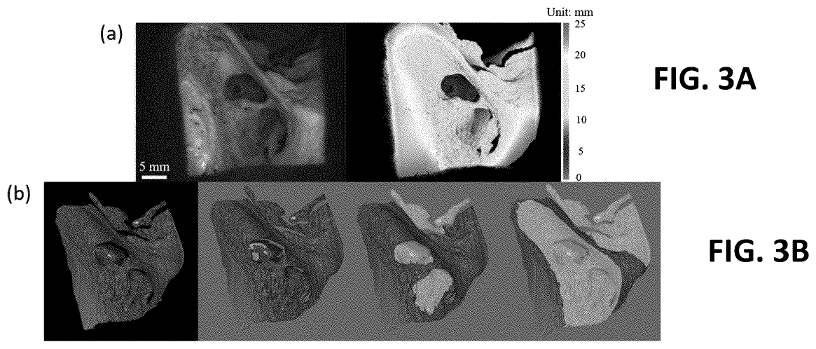

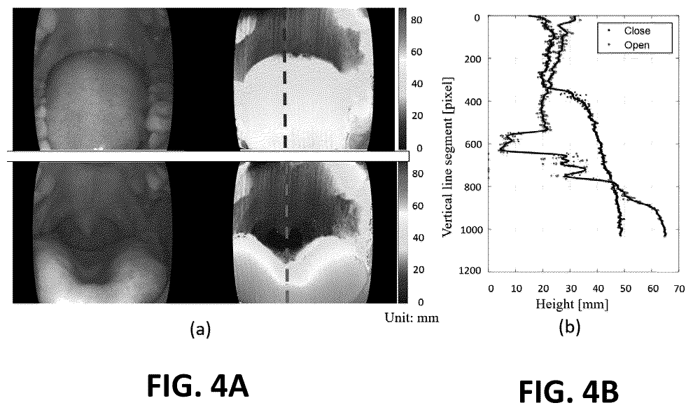

To validate the system performance for imaging biological samples, 3D imaging of a human dried temporal bone, and an in vivo human mouth cavity was performed. The depth reconstruction height maps in both 2D and 3D are displayed in FIGS. 3A, 3B, 4A, and 4B, respectively.

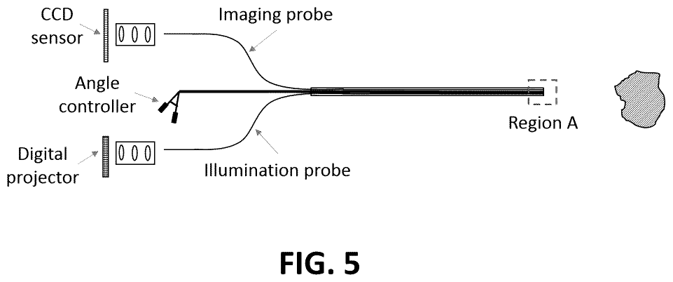

FIGS. 3A and 3B display the 3D structure of a human dried temporal bone, specifically in the cochlear section. FIG. 3A shows the white reflectance image (left) of the bone sample with its corresponding gradient color height image (right). FIG. 3B shows three cavity sections at different depth levels of 5 mm, 10 mm and 18 mm above the zero height. The natural shape and pattern of the cochlear cavity are well indicated in the both 2D and 3D height maps. The different depth sections indicated in FIG. 3B are separated by the height gaps of 5 mm and 8 mm, and are also well correlated with the physical distance of 5.013±0.0416 mm and 7.983±0.040 mm, respectively. FIGS. 3A and 3B illustrate image views of human dried temporal bone reveals cochlear section with its height map in 2D, as illustrated in FIG. 3A, and its corresponding 3D images at different section planes, as illustrated in FIG. 5B. Scale bar 5 mm.

The next experiment focuses on the dynamic capturing of a mouth cavity in both closed and opened state. As shown in FIGS. 4A and 4B with details such as tonsil and the inner back wall revealed, the FPP technique can be used with endoscopes even with dynamic objects. However, specular reflectance introduces noise that can be solved by using a cross-polarized light illumination source. In 3D imaging of live tissue, a compensation between the camera integration time and the image acquisition time should be made for motion artifact minimization as well as for accuracy maintenance. In the mouth cavity experiment, the camera integrating time is 200 millisecond and the image acquisition time is less than 2 seconds without the use of GPU on the workstation. The main reasons for the relatively slow imaging speed are due to the low illumination level and the low sensitivity of the camera used. FIG. 4A illustrates image views of a human mouth cavity reflectance image with its corresponding height map when the inner cavity is close (above row) and open (below row), and FIG. 4B illustrates a height of segmented sections for both cases along the dashed lines.

The system's relative sensitivity is about 0.1% with a depth FOV of 2 cm. The proposed imaging system is the foundation for real time 3D endoscope with the use of parallelization and the integration of both illumination and imaging scopes into a single scope. Besides 3D capturing, endoscopic guided imaging can be integrated with other tissue analysis techniques such as speckle imaging and multispectral imaging to evaluate tissue perfusion kinetics and classify tissue types based on its spectral signatures.

Besides the setup as described above several other embodiments meet the relative height accuracy of 0.1% and the overall housing diameter of about 10 mm or less. The designs introduce the optical components for structure illumination and imaging site and simplify the use of two separate endoscopes, therefore, support the incision minimization and systemic stability.

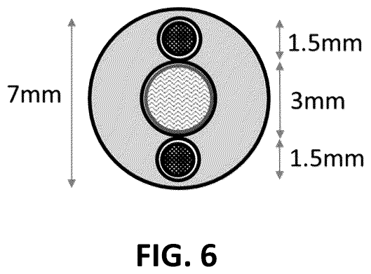

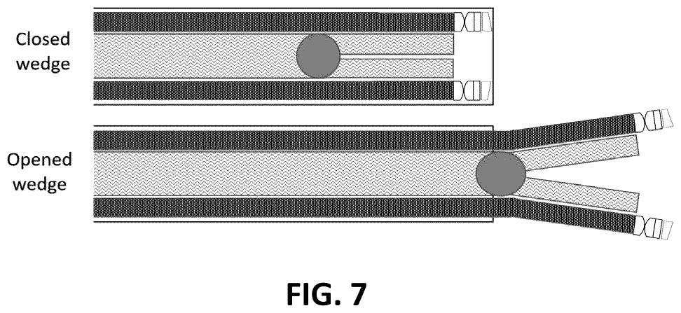

The first design utilizes two flexible imaging probes for illumination and imaging purpose, housed in two smaller ports in a rigid tube as described in FIGS. 5 and 6. The flexible fiber bundles are used to transmit the generated fringe patterns from the digital projectors to the object and to collected reflected light bounced back from the object to the CCD sensor. For the maintenance of height accuracy, the separation between two fibers is restricted to a set distance. Here an angle-controller flexible probe is included which aims to fix this separation. This flexible probe functions similarly as a biopsy probe, i.e. the opening angle (wedge angle) at the distal end of the angle controller is varied by squeezing the controller handle. After the wedge angle is determined, the angle is locked in the same way as the hemostat locking mechanism. FIG. 6 describes the housing for both fiber probes and the angle controller with two ports for the fiber probes (identical 1.5 mm in diameter) and the middle bigger port for the angle controller (diameter of 3 mm). The total housing diameter is 7 mm, however, this measurement is tentative if a smaller design of the fiber imaging scope and the angle-controller probe are employed. The overall diameter requirement for the tubic casing is from 4 mm to 10 mm. FIG. 5 illustrates a schematic diagram of an embodiment with a flexible fiber-based endoscope. Region A is detailed in FIG. 7. FIG. 6 illustrates a cross-sectional view of the tubing containing the fibers and angle-controller flexible probe.

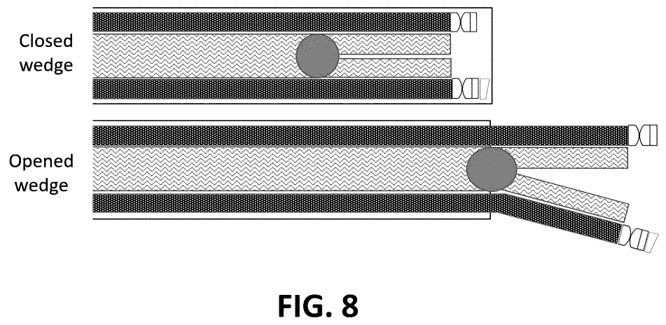

For the purpose of pattern projection and image collection, both illumination and imaging fibers are attached with a pair of achromatic doublets as illustrated in FIGS. 7 and 8. The lens combination aims to deliver and collect light rays in telescopic view (An alternative solution for the achromatic lens is grin lens). In addition, a wedge prism for angle deviation control is also used (Some alternative options for the prism are fiber polishing or tapering). FIGS. 7 and 8 indicate the mechanism of the scope when using the angle controller. When the angle controller handles are at rest (not being squeezed), the angle wedge is closed and both fibers and the controller probe are inside the housing tube. When the angle controller handles are squeezed, the wedge is opened, both fibers with its optics and the wedge arms are exposed while the two arms of the wedge is opened at a desired angle, supporting the separation between two fiber scopes.

FIG. 7 illustrates a partially sectional view of Region A of FIG. 5 with two angle control cases (when the wedge is closed and opened). At the distal end of each flexible fiber, a combination of prism and doblet lens is used for ray deviation and illumination and imaging purposes. (Alternative solutions for deviation prism are using taper or fiber polishing). In FIG. 7, both arms of the wedge are deviated, an alternative solution is only the lower arm of the wedge is moved (FIG. 8). In this design, only one angle deviation prism is needed and the wedge opening angle is double as comparison to the FIG. 7 scenario. FIG. 8 illustrates a partially sectional view of another embodiment of region A of FIG. 5. FIG. 8 is a variation on FIG. 7, where only the lower arm of the wedge is moved, therefore the deviation angle is double; however, only 1 prism is used in comparison with the setup in FIG. 7.

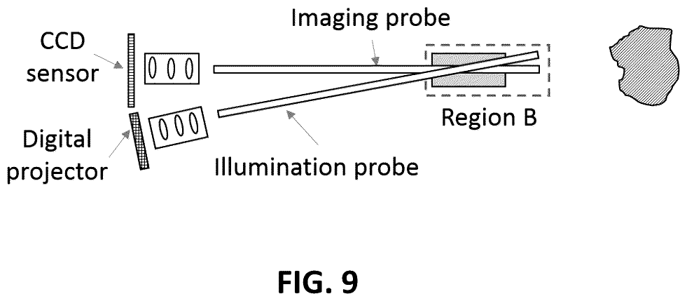

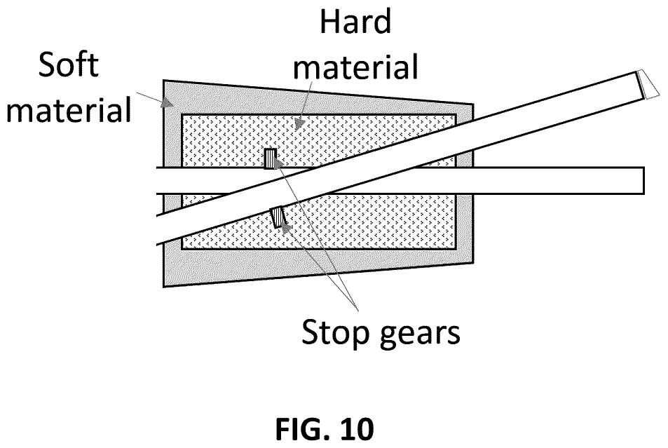

Another embodiment is an alternative way for using flexible scope by utilizing the commercially available rigid surgical endoscope as indicated in FIG. 9. In order to fix the scope separation, an angle fixation adaptor as illustrated in FIG. 10 can be located at the abdomental wall (where the trocar in a minimally invasive surgery is placed), the two scopes are subsequently slided into this adapter and fixed using two stop gears embedded on the inner wall of the adaptor. For friction reduction between the adaptor and the abdomen tissue at the entrance, the adaptor is covered by a tubing with soft medical-graded polymer materials such as silicone, butyl, polyisoprene and styrene butadiene rubber. Inasmuch as the telescopic optics are available inside the ridged scope, a wedge prism is attached in front of a rigid scope to deviate the angle. FIG. 9 illustrates a schematic diagram of another embodiment with two commercially available rigid scopes with a fixation adapter. Region B is detailed in FIG. 10. FIG. 10 illustrates a partially sectional view of region B of FIG. 9). FIG. 10 illustrates a cross-section of the fixation adapter with the two stop-gear for the translational and rotational fixation.

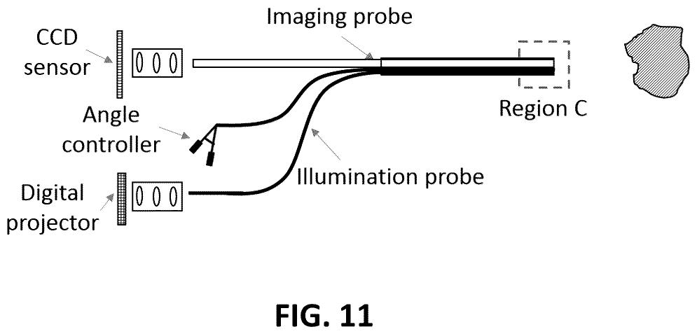

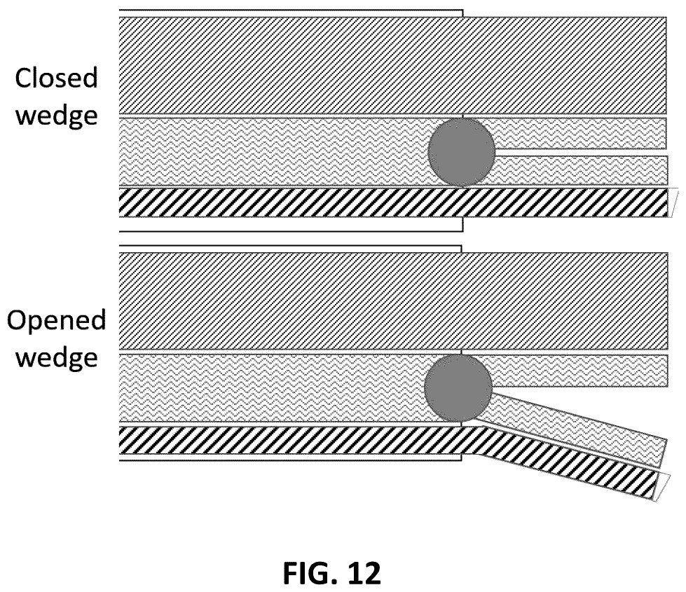

Another embodiment allows the use of the common medical-graded scopes in both rigid and flexible forms. Where the illumination and imaging tasks can be performed in either of the scope. FIG. 11 demonstrates an example where the rigid scope is used as imaging probe and the flexible scope as the illumination probe. The use of a flexible scope enables the housing minimization and the housing design is similar as the schematic provided in FIG. 6. Moreover, the required separation between the two scopes (FIG. 12) can be deviated based on the similar angle controller as explained in FIG. 8. In this design, both scopes are equipped with the embedded optics adequated for the illumination and imaging purpose, however, the deviated scope is attached with a wedge prism to the object site for the triangulation purpose. FIG. 11 illustrates a schematic diagram of another embodiment with one rigid scope and a flexible medical graded scope with the angle controller. FIG. 12 illustrates a partially sectional view of region C in FIG. 11. Two cases of angle control functions similarly as in FIG. 8 with the controlled lower arm deviates angle of the flexible scope.

In addition, the above-mentioned design, setup in FIG. 11 can be modified using a combination of two flexible medical-graded scopes. In that, the setup and angle deviation mechanism are similarly as in FIGS. 5, 7 and 8.

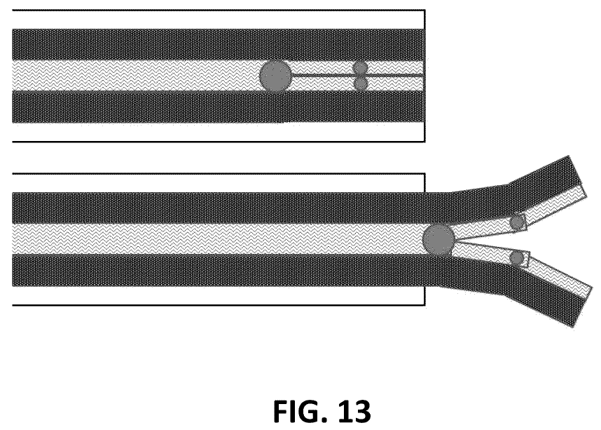

As an extension of the angle controller design, a design with multiple joints as indicated in FIG. 13 can be used to minimize tissue damage in case with small accessing entrance to the target site. The number of joints can be more than two joints as demonstrated in FIG. 13. Besides the manual mechanical function of the angle controller as described, other actuation mechanism using piezo electric, magnetic or other hydraulic fluid pressure can also be used. FIG. 13 illustrates a partially sectional view of an extended wedge arm with multiple joints to access small space region.

The use of the common rigid/flexible scope for the 3D reconstruction can also be used to characterize tissue biological properties using multispectral or speckle imaging technique. A customized spectral light source can be couple into the light port of the scope or into a separate light pipe (for a customized fiber bundle scheme) which support the illumination on the object. Tissue information such as its textures, tissue classification, tissue thickness, vascular structure, blood perfusion and blood flow can be deduced based on spectral analysis.

The present invention can be carried out and/or supported using a computer, non-transitory computer readable medium, or alternately a computing device or non-transitory computer readable medium incorporated into the imaging device. Indeed, any suitable method of calculation known to or conceivable by one of skill in the art could be used. It should also be noted that while specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

The computing device can be a special computer designed specifically for this purpose. The computing device can be unique to the present invention and designed specifically to carry out the method of the present invention. Imaging devices generally have a console which is a proprietary master control center of the imager designed specifically to carry out the operations of the imager and receive the imaging data created by the imager. Typically, this console is made up of a specialized computer, custom keyboard, and multiple monitors. There can be two different types of control consoles, one used by the operator and the other used by the physician. The operator's console controls such variables as the thickness of the image, the amount of tube current/voltage, mechanical movement of the patient table and other radiographic technique factors. The physician's viewing console allows viewing of the images without interfering with the normal imager operation. This console is capable of rudimentary image analysis. The operating console computer is a non-generic computer specifically designed by the imager manufacturer for bilateral (input output) communication with the scanner. It is not a standard business or personal computer that can be purchased at a local store. Additionally this console computer carries out communications with the imager through the execution of proprietary custom built software that is designed and written by the imager manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

What is claimed is:

1. A device for 3D imagery comprising:
an endoscopic probe comprising an imaging probe and an illumination probe configured for fringe projection profilometry (FPP) and configured to provide a wide field of view (FOV);
an angle controller disposed between the imaging probe and the illumination probe, wherein the angle controller is configured to adjust an angle of separation between the illumination probe and the imaging probe;
a CCD sensor; and
a digital projector.

2. The device of claim 1 wherein the angle controller takes the form of a flexible probe.

3. The device of claim 1 wherein the angle controller sets a distance for separation between the imaging probe and the illumination probe.

4. The device of claim 1 further comprising a housing for the imaging probe, the illumination probe, and the angle controller.

5. The device of claim 1 further comprising a non-transitory computer readable medium programmed to execute a flexible camera calibration method for 3D reconstruction in free space to provide an optimal fringe pattern for an inner tissue profile.

6. The device of claim 1 wherein the imaging probe and the illumination probe each have a diameter ranging from 500 um to 10 mm.

7. The device of claim 1 further comprising a digital micromirror device (DMD).

8. The device of claim 7 wherein the DMD is configured to project a fringe pattern.

9. The device of claim 1 further comprising a minimum 15° angle between the imaging and illumination probe.

10. The device of claim 1 further comprising synchronizing structured patterns from the DMD with the imaging camera.

11. A method for a 3D image of a region of interest comprising:
providing a wide field of view (FOV) with an imaging probe, such that quantitative depth information is addressed;
illuminating the region of interest;
projecting a fringe pattern; and
capturing the 3D image of the region of interest.

12. The method of claim 11 further comprising using an illumination probe for illuminating the region of interest.

13. The method of claim 12 further comprising setting a distance of separation between the imaging probe and the illumination probe.

14. The method of claim 13 further comprising setting the distance of separation to be a minimum of a 15° angle.

15. The method of claim 11 further comprising executing the method in conjunction with a non-transitory computer readable medium.

16. The method of claim 15 further comprising programming the non-transitory computer readable medium to execute a flexible camera calibration method for 3D reconstruction in free space to provide an optimal fringe pattern for an inner tissue profile.

17. The method of claim 11 further comprising using a digital micromirror device (DMD) to project the fringe pattern.

18. The method of claim 17 further comprising using a coordinate transform to correspond each image point to each respective point on the DMD via the collected fringe patterns.

19. The method of claim 11 further comprising using a CCD camera to capture the 3D image of the region of interest.

20. The method of claim 11 further comprising providing tissue profilometry.
